(19) United States
(12) Patent Application Publication
(10) Pub. No.: US 2010/0233247 A1
(43) Pub. Date: Sep. 16, 2010

(54) Dressing Material Comprising Active Substances

(76) Inventor: Sabine Hohmann, Heidelberg (DE)

Correspondence Address: BIRCH STEWART KOLASCH & BIRCH, PO BOX 747, FALLS CHURCH, VA, 22040-0747, US

(21) Appl. No.: 11/991,810

(22) PCT Filed: Aug. 1, 2006
(86) PCT No.: PCT/EP2006/064887

§ 371 Date: Apr. 28, 2008

(30) Foreign Application Priority Data
Sep. 13, 2005 (EP) 05019854.8

(51) Int. Cl. A61L 15/16; A61K 36/28; A61K 36/22; A61K 36/75; A61K 36/42; A61K 36/38; A61K 35/64; A61K 36/736; A61K 36/00; A61P 19/02; A61P 19/04

(52) U.S. Cl. 424/445; 424/725; 424/730; 424/539; 424/735; 424/778

(57) ABSTRACT

The present invention relates to a dressing material comprising a support material and at least one active substance from in each case Arnica montana, Rhus toxicodendron and Ruta graveolens. The invention further relates to a medicament comprising this dressing material and to the use of this dressing material for producing a medicament for the treatment of disorders or impairments of the musculoskeletal system. The dressing material is preferably employed in this connection as tape dressing.

The present invention relates to a dressing material comprising a support material and at least one active substance from in each case Arnica montana, Rhus toxicodendron and Ruta graveolens. The invention further relates to a medicament comprising this dressing material and to the use of this dressing material for producing a medicament for the treatment of disorders or impairments of the musculoskeletal system. The dressing material is preferably employed in this connection as tape dressing.

Injuries of the musculoskeletal system, e.g. capsular ligament injuries, myotendinous injuries, bone injuries or else degenerative processes, can be treated by partial or complete immobilization of the affected parts of the body. Complete immobilization, e.g. by plaster casts, is well known in the prior art. One disadvantage of this treatment method is, however, that the affected parts of the body must often be immobilized over a prolonged period, and the parts of the body may experience secondary damage through the mode of immobilization, e.g. plaster cast, such as blood flow impairments, reduced lymphatic drainage or else tissue atrophies. Besides complete immobilization of affected parts of the body, there are now also methods for partial immobilization. Particularly important in this connection are the tape dressings through which various injuries can be induced to heal in a short time without complete immobilization (e.g. by a plaster) being necessary. At least some of the secondary damage which may occur through the known measures for complete immobilization of parts of the body can thus be effectively avoided. The tape dressing supports and relieves damaged portions, e.g. of a joint, for movements, permits functional load-bearing to a limited extent and thus avoids extreme movements which, in some circumstances, might lead to further damage to various portions of the joint. However, it makes functional follow-up treatments possible, e.g. after a joint operation. Tape dressings have in this connection not only a stabilizing effect as passive outer sheath, but also activate groups of muscles via stimulation of mechanoreceptors in the skin and proprioreceptors of muscles and tendons. The contraction of the activated muscle groups brings about an additional stabilization, also referred to as proprioceptive effect. Medical care is thus now inconceivable without tape dressings. Nevertheless, some of the secondary damage mentioned above in connection with dressings which bring about complete immobilization of a part of the body can also occur with tape dressings. These include in particular damage due to inadequate lymphatic drainage, blood supply or tissue atrophies.

It is thus an object of the present invention to provide measures by which the aforementioned disadvantages can be avoided, simultaneously enabling an improved healing process for damaged parts of the body's musculoskeletal system.

The object is achieved by the embodiments described in the claims and subsequently.

The invention therefore relates to a dressing material comprising a support material and at least one active substance from in each case Arnica montana, Rhus toxicodendron and Ruta graveolens. The present invention further relates to a dressing material comprising a support material and at least one active substance from in each case Flos cathaimi, Ruta graveolens and Rhus delavayi.

The present invention additionally relates also to dressing materials which comprise a support material, preferably as described above, and have at least one further active substance which is selected from the group consisting of: (i) active substances from Bryonia alba, Bryonia dioica, mixtures of cherry plum flowers, flowers of white clematis, flowers of impatiens glandulifera, flowers of the yellow sunrose and flowers of the Star of Bethlehem (commercially available as rescue remedy mixture), Hypericum perforatum (St. John's wort), Apis mellifica, Calendula, Agaricus muscarius, Anacardium orientate, Lendium palustris, Bellis perennsis, Hamamelis viriginica and (ii) the active substances Strontium carbonicum, Acidum sulforicum and diclofenac. Diclofenac is a well-known medicament with the chemical name sodium [2-(2,6-dichloroanilino)phenyl]acetate.

A dressing material in the context of the present invention is a flexible material which can be applied to the skin of the body on damaged parts of the body's musculoskeletal system. The flexibility of the dressing material should be selected so that on the one hand it permits guided, assisted movement of the affected parts of the musculoskeletal system but, on the other hand, sufficiently fixes the latter in order to avoid unwanted or extreme movements such as overstretching. The dressing material of the invention may be composed of one or more materials. The dressing material comprises at least one support material and an active substance or an active substance combination as described above. The active substance can in this case be applied directly to the support material or be coupled to an auxiliary substance which is applied to the support material. Besides the support material and the active substances, the dressing material may thus also comprise auxiliary substances. Suitable auxiliary substances include pharmaceutically acceptable solvents for the active substances, substances which influence the elasticity or rigidity of the support material, but also substances which are required for attaching the dressing material to the skin, such as, for example, adhesive. The dressing material may have any geometric shape. It is preferably in the form of a rectangular or circular plaster or, particularly preferably, as rectangular tape. The dressing material of the invention is preferably air-permeable, water-repelling and consists of hypoallergenic materials. It is possible by selection of suitable colors, preferably red, blue, yellow additionally to have an influence in an energetic way on the events in the body and the healing process. It is also possible according to the invention to employ as dressing materials all commercially available plasters, dressings or tape dressings.

The term “support material” means in the context of the present invention a woven or unwoven material which may be composed of one or more different constituents. The basic structure of the support material is preferably composed of a natural or synthetic polymer, e.g. a polyester, or a polycondensate, e.g. a polyhydroxyalkanoate. Suitable substances for the basic structure of the support material are known in the prior art. The support material particularly preferably comprises a mixture of natural constituents, preferably cotton or rubber, and synthetic constituents, preferably acrylic. Further substances, e.g. the aforementioned auxiliary substances, can be introduced into the support material. The support material should further be of such a nature that it can enter into a connection, either directly or via auxiliary substances, with the aforementioned active substances or active substance combinations. The support material should likewise preferably be able to enter into a connection with an adhesive, as disclosed elsewhere in the description.

The term “at least one active substance” refers both to isolated active substances and to active substance mixtures. An active substance as used herein is a chemical compound which can be obtained from one of the aforementioned plants or a plant part, e.g. seeds, leaves or stems, thereof. An active substance mixture in the context of the present invention is a mixture of such chemical compounds. The mixture may in this case be produced by mixing appropriate individual active substances or be provided by obtaining different active substances together from the plants or plant portions. In the context of the present invention, an active substance mixture is preferably obtained from in each case Arnica montana, Rhus toxicodendron and Ruta graveolens by extraction with a solvent consisting of an alcohol and water. For this purpose, firstly in each case healthy plants are harvested, cleaned and then freshly comminuted. The comminuted plant pulp is then mixed with the solvent and incubated preferably in the dark, so that the active substances can be dissolved out of the comminuted plant fragments by the solvent. An original extract is then obtained by subsequent separation of the comminuted plant fragments from the solvent/active substance mixture. The separation can take place for example by filtration, sedimentation or centrifugation. The original extract is preferably obtained by filtration. The solvent used preferably consists of 90% alcohol and 10% water. The alcohol is particularly preferably ethanol. Details of the active substance formulation and production of the active substances which are to be employed in the context of the present invention are to be found in “Deutsches Homöopathisches Arzneibuch”, Schwabe, Leipzig, 1901, (HAB). Products which comprise the correspondingly produced active substances and/or active substance mixtures are commercially available. Extracts from Arnica montana are preferably obtained from the dried underground portions of the plant or the above-ground portions at flowering time according to Rules 4a and 17 of the HAB. Extracts from Rhus toxicodendron are preferably obtained from young, as yet unlignified shoots according to Rules 2a and 7 of the HAB. Extracts from Ruta graveolens are preferably obtained from the fresh, above-ground parts at flowering time according to Rules 3a and 7 of the HAB. The original extract produced by the processes described above is preferably employed as dilute extract in the dressing material of the invention. The diluent employed in this case is likewise an alcohol/water mixture, preferably as described above. The dilute extract is preferably produced by shaking, as described in the HAB.

Alternatively, active substances which cannot be dissolved in alcohol, distilled water from the raw material (e.g. plant leaves), can initially be triturated to the 3rd/4th power. In the trituration, the raw material is ground and scraped up 3× with lactose per potentiation step in a mortar, and the ratio between raw material and lactose should be about 1:100. The previously described extracts or triturations are preferably formulated for local use.

The active substances from the plants or plant constituents of Flos cathaimi, Rhus Delavayi, Bryonia alba, Bryonia dioica, St. John's wort, Apis melifica, Calendula, Agaricus muscarius, Anacardium Orientale, Lendium palustris, Bellis perennsis and Hamamelis virginica can likewise be obtained in accordance with the HAB protocols. It is possible and particularly preferred for active substances to be obtained from Bryonia alba or dioica in accordance with Rule 2a of the HAB. Active substances from Agaricus muscarius can preferably be obtained according to Rule 3a of the HAB. Active substances from Apis melifica can preferably be obtained according to Rule 4b of the HAB.

The active substances and active substance mixtures are particularly preferably formulated as ointments or creams. Besides the aforementioned extracts or triturations, the ointments or creams preferably also comprise woolwax, woolwax alcohol ointment (consisting of cetylstearyl alcohol, woolwax alcohols and white Vasiline), high-viscosity paraffin and water. The ointments normally comprise the extract or the trituration with the active substances in a concentration of 5-20% (w/w), preferably 10% (w/w). Instead of ointment form, the aforementioned extracts or triturations can also be applied to the support material in any other form suitable for local administration, e.g. as gel. However, the precondition is that this administration form allows bioavailability of the active substances on local, dermal administration.

It has been found in the context of the present invention that a dressing material which comprises the aforementioned constituents has the therapeutic advantages of a tape dressing known in the prior art, but at the same time can avoid its disadvantages. The additional application of active substances, e.g. from Arnica montana, Rhus toxicodendron and Ruta graveolens, and the local care, made possible thereby, of the section, provided with the dressing material, of the musculoskeletal system brings about efficient lymphatic drainage, increased blood supply (especially venous activation), a stabilization of the damaged structure with retention of good mobility, and avoidance of muscle atrophy, and a therapy-promoting influence on cutaneous and pain receptors, muscle and muscular attachments, and of the capsular and ligamentous system. Injuries and impairments of parts of the musculoskeletal system such as muscular pain syndromes, muscle cramps, joint pain and joint sprains, tendon and ligament complaints, rheumatic complaints, contusions, overstretchings, muscle fiber tears, ruptured ligaments, strains, fresh injuries with pronounced hematomas, finger sprains, skier's thumb, tennis and golf elbows, impingment syndromes, insertion tendopathies, adductor injuries, knee joint effusions, knee joint complaints, Achilles tendon complaints, heel spurs, ankle complaints, forefoot and metatarsal pain (longitudinal and transverse arch), wrist complaints, cervical spine, thoracic spine or longitudinal spine complaints, migraine or lymphedemas can, thanks to the present invention, be treated effectively and without side effects or secondary damage. In the case studies underlying the present invention, it was found that the advantageous effects of a dressing material, e.g. of a tape dressing, can be combined synergistically with the therapeutic effects of the aforementioned active substances, with, in addition to the synergism found, it also being observed simultaneously that there was a drastic reduction in the secondary damage which may be induced by a tape dressing.

In a preferred embodiment of the dressing material of the invention, the at least one active substance from Arnica montana is a dilute extract which consists of a partial extract in 10¹² parts of a pharmaceutically acceptable solvent, preferably an alcohol/water mixture as described above. Such a dilute extract is also referred to as Arnica D12 in the prior art and is commercially available.

In a preferred embodiment of the dressing material of the invention, the at least one active substance from Rhus toxicodendron is a dilute extract which consists of a partial extract in 10¹² parts of a pharmaceutically acceptable solvent, preferably an ethanol/water mixture as described above. Such a dilute extract is also referred to as Rhus toxicodendron D12 in the prior art and is commercially available.

In a preferred embodiment of the dressing material of the invention, the at least one active substance from Ruta graveolens is a dilute extract which consists of a partial extract in 10¹² parts of a pharmaceutically acceptable solvent, preferably an ethanol/water mixture as described above. Such a dilute extract is also referred to as Ruta graveolens D12 in the prior art and is commercially available.

In a further preferred embodiment of the dressing material of the invention, this includes an adhesive for attaching the dressing material to the skin. The compositions which can be used as “adhesive” in the context of the present invention are those which mediate a reversible connection between the skin and the dressing material. These adhesives are preferably bonding agents. These bonding agents are preferably compatible with skin and do not cause any other body reactions either, such as contact allergies. Suitable bonding agents for dressing materials such as plasters or tape dressings are well known in the prior art. The dressing material may be impregnated with the adhesive or coated therewith, preferably on one side. The dressing material is preferably not completely provided with the adhesive.

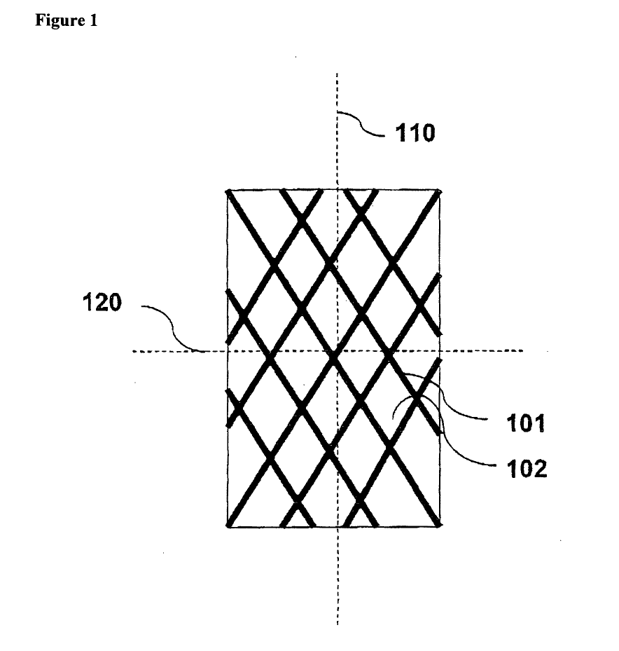

In a preferred embodiment of the dressing material, the adhesive is applied along the margins of rhombic areas (102), preferably in the form of splinters (101), so that the rhombic areas of the rhombi formed by the margins remain free of adhesive. A corresponding dressing material is shown in FIG. 1. The rhombic areas are then most preferably provided with the active substances or active substance mixtures. The dressing material can in this case be impregnated with solutions of the corresponding active substances or be coated therewith, with coating being preferred.

In a further preferred embodiment of the dressing material, the adhesive is applied two-dimensionally to the support material. In this embodiment, the active substances are preferably introduced directly into a suitable adhesive before the latter is applied to the support material. Suitable adhesives which do not impair the pharmaceutical properties of the active substances are known in the prior art. Alternatively, the active substances or active substance mixtures can be applied subsequently, e.g. as film onto an adhesive which has already been applied to the support material.

In a further preferred embodiment of the dressing material of the invention, the latter is up to 95% extensible along a first axis (110). The course of the first axis of the dressing material is depicted in FIG. 1. The first axis in the context of the invention is preferably the longitudinal axis of the dressing material and is distinguished, when the dressing material is applied, by running parallel to an imaginary axis along the skin surface and, if a dressing material with rectangular shape, e.g. a tape, is used as dressing material, by running parallel to the longer of the sides of the rectangle.

In a further preferred embodiment of the dressing material of the invention, the dressing material is no more than 5% extensible along a second axis (120). The second axis in the context of the invention is preferably the transverse axis of the dressing material and is distinguished, when the dressing material is applied, by running parallel to an imaginary axis along the skin surface and, if a dressing material with rectangular shape, e.g. a tape, is used as dressing material, by running parallel to the shorter of the sides of the rectangle.

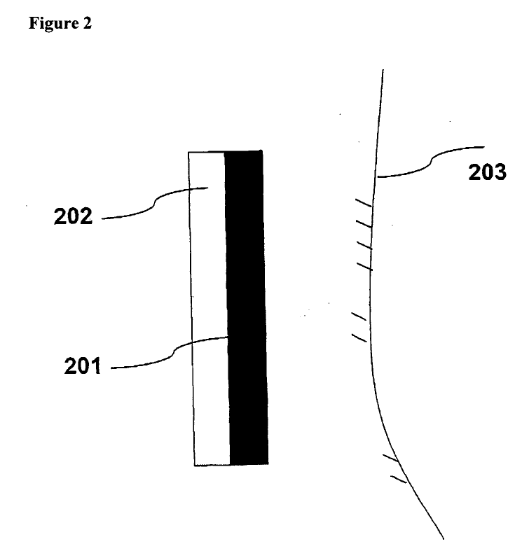

Also preferred in the context of the present invention is a dressing material where the support material includes a first support material (201) which is to be brought into contact with the skin, and a second support material (202) for the outside of the dressing. The first support material (201) preferably comprises zinc oxide, rubber and acrylic, with preferably 1 to 10%, particularly preferably 5% (w/w), zinc oxide, preferably 1 to 10%, particularly preferably 5% (w/w), rubber and preferably 80 to 98%, particularly preferably 90% (w/w), acrylic being present. The proportions of zinc oxide, rubber and acrylic in the first support material should, however, always add up to 100% (w/w) in the context of this preferred embodiment. The second support material (202) is preferably cotton, particularly preferably pure cotton (100%). A preferred support material in the context of the invention is shown in FIG. 2 and described in the exemplary embodiment.

The invention also relates to a medicament comprising a dressing material of the invention as described above. The dressing material is preferably packaged sterile in the case of the medicament formulation. If a tape is to be employed as dressing material, the dressing material is preferably provided as medicament rolled up on a roll. The dosage of the active substances included in the dressing material has already been described above. The medicament is particularly preferably formulated so that it can be employed as tape dressing. For such a medicament, a rectangular tape of dressing material, preferably 5 to 15 m long, most preferably 10 m long, is provided on a roll.

The invention finally relates to the use of a dressing material of the invention as described above for producing a medicament for the treatment of disorders or impairments of the musculoskeletal system, preferably muscular pain syndrome, muscle cramps, joint pain and joint sprains, tendon and ligament complaints, rheumatic complaints, contusions, overstretchings, muscle fiber tears, ruptured ligaments, strains, fresh injuries with pronounced hematomas, finger sprains, skier's thumb, tennis and golf elbows, impingment syndrome, insertion endopathy, adductor injuries, knee joint effusions, knee joint complaints, Achilles tendon complaints, heel spurs, ankle complaints, forefoot and metatarsal pain (longitudinal and transverse arch), wrist complaints, cervical spine, thoracic spine or longitudinal spine complaints, migraine or lymphedemas. The dressing material is in this case preferably employed as tape dressing.

The figures show:

FIG. 1: view of the skin contact area of a support material of the invention, showing margins of the rhombic areas (101) which are to be provided with adhesive, the adhesive-free rhombic areas (102), and the first (longitudinal) axis (110) and the second (transverse) axis (120).

FIG. 2: the figure shows a cross section through a support material of the invention which consists of a first support material (201) and of a second support material (202). The first support material (201) is in this case to be brought into contact with the skin surface (203).

The invention is now illustrated by means of the exemplary embodiments. The exemplary embodiments are not intended in this connection to restrict the scope of protection.

### EXAMPLE 1

### Tape Dressing Material

A tape about 10 m long and about 4.5 cm wide consists of a surface material and of a material applied on the skin side. The surface material is 100% cotton. The material on the skin side consists of 5% zinc oxide, 5% rubber and 90% acrylic. An adhesive is applied in strips to the material on the skin side, the strips being applied so that they form the margins of rhomboidal areas. Dilute extracts of Arnica montana, Rhus toxicodendron and Ruta graveolens are applied inside these rhombic areas. The extracts are Arnica D12, Rhus toxicodendron D12 and Ruta graveolens D12. The dressing material is processed so that it is air-permeable and water-repellent. The dressing material permits 95% extension longitudinally and 5% extension transversely. The substances used for the dressing material are hypoallergenic. The dressing material is kept in the colors red, blue, yellow.

The dressing material is employed as tape dressing for injuries and impairments of the musculoskeletal system. It has already achieved very successful healing in various ways. In particular, lymphatic drainage and a better blood supply of the affected parts of the musculoskeletal system have been achievable, and muscle atrophies avoided.

### EXAMPLE 2

### Case Study

Patient: male, 30 years, sportsman
History: suffered a ruptured Achilles tendon while playing tennis
Diagnosis: a partial rupture was found with the aid of an ultrasound investigation
Previous therapy: Adimed shoe with heel wedges for 4-6 weeks (during this time, wedge altered to the neutral-null position of the ankle joint)

Patient still complained for weeks after the healing phase that the foot was thicker and of pain when starting to move in the morning and feeling of instability.

Therapy with Tape Dressing Material from Example 1:

The pain when starting to move in the morning and the edema were eliminated within 2 days by applying the dressing. The dressing was changed after 1 week and completely removed after the 2nd week. The patient was able to engage in his sporting activities again as normal.

The effect of the active substances on the tape dressing had a positive influence on blood and blood vessels and lymphatic system. Tissue regeneration is improved through the increase in the blood flow (which is, after all, not optimally supplied due to the trauma when the muscle-capsule-ligament system is injured). Application of the tape dressing, which adapts to the skin movements through its flexibility, activated the skin and pain receptors and the circulatory system of the patient with every movement, so that the active substances were able to act locally. Transport away of tissue fluid was increased. The circulation was distinctly accelerated and thus the healing process was shortened. A faster reduction in pain allows faster load-bearing to be possible, and thus in turn an increase in the circulatory system was brought about and, at the same time, build up of muscle was assisted.

1-19. (canceled)

20. A dressing material comprising a support material and at least one active substance from in each case Arnica montana, Rhus toxicodendron and Ruta graveolens.

21. The dressing material of claim 20, where the at least one active substance from Arnica montana is a dilute extract which consists of a partial extract in 10¹² parts of a pharmaceutically acceptable solvent (Arnica D12).

22. The dressing material of claim 20, where the at least one active substance from Rhus toxicodendron, a dilute extract, which consists of a partial extract in 10¹² parts of a pharmaceutically acceptable solvent (Rhus toxicodendron D12).

23. The dressing material of claim 20, where the at least one active substance from Ruta graveolens is a dilute extract which consists of a partial extract in 10¹² parts of a pharmaceutically acceptable solvent (Ruta graveolens D12).

24. A dressing material comprising a support material and at least one active substance from in each case Flos cathaimi, Ruta graveolens and Rhus delavayi.

25. A dressing material comprising a support material and at least one further active substance selected from the group consisting of: Bryonia alba, Bryonia dioica, mixtures of cherry plum flowers, flowers of white clematis, flowers of impatiens glandulifera, flowers of the yellow sunrose and flowers of the Star of Bethlehem, Hypericum perforatum, Apis mellifica, Calendula, Strontium carbonicum, Agaricus muscarius, Anacardium orientale, Lendium palustris, Bellis percunis, Acidum sulforicum, Hamamelis virginica and diclofenac.

26. The dressing material of claim 20, which includes an adhesive for attaching the dressing material to the skin.

27. The dressing material of claim 26, where the adhesive is applied along the margins of rhombic areas (102), and the rhombic areas remain free of adhesive.

28. The dressing material of claim 27, where the rhombic areas are provided with the at least one active substance from in each case Arnica montana, Rhus toxicodendron, Ruta graveolens.

29. The dressing material of claim 20, where the dressing material is up to 95% extensible along a first axis (110).

30. The dressing material of claim 20, where the dressing material is no more than 5% extensible along a second axis (120).

31. The dressing material of claim 20, where the support material includes a first support material (201) which is to be brought into contact with the skin, and includes a second support material (202) for the outside of the dressing.

32. The dressing material of claim 31, where the first support material (201) comprises zinc oxide, rubber and acrylic.

33. The dressing material of claim 32, where 1 to 10% (w/w) zinc oxide, 1 to 10% (w/w) rubber and 80 to 98% (w/w) acrylic are present.

34. The dressing material of claim 32, where 5% (w/w) zinc oxide, 5% (w/w) rubber and 90% (w/w) acrylic are present.

35. The dressing material of claim 31, where the second support material (202) comprises cotton.

36. A method for treating muscular pain syndrome, muscle cramps, joint pain and joint sprains, tendon and ligament complaints, rheumatic complaints, contusions, overstretchings, muscle fiber tears, ruptured ligaments, strains, fresh injuries with pronounced hematoma, finger sprains, skier's thumb, tennis and golf elbows, impingment syndrome, insertion endopathy, adductor injuries, knee joint effusions, knee joint complaints, Achilles tendon complaints, heel spur, ankle complaints, forefoot and metatarsal pain (longitudinal and transverse arch), wrist complaints, cervical spine, thoracic spine or longitudinal spine complaints, migraine or lymphedemas in a subject comprising applying a dressing material as claimed in any of claims 1 to 16.

37. The method of claim 36, wherein the dressing material is applied as tape dressing.

38. The dressing material of claim 33, where 5% (w/w) zinc oxide, 5% (w/w) rubber and 90% (w/w) acrylic are present.
